(19) United States
(12) Patent Application Publication
(10) Pub. No.: US 2024/0423893 A1
(43) Pub. Date: Dec. 26, 2024

(54) CARRAGEENAN AND STARCH TEXTURIZER BASED NATURAL DEODORANT

(71) Applicant: CARGILL, INCORPORATED, Wayzata, MN (US)

(72) Inventor: Smita FULZELE, Plymouth, MN (US)

(73) Assignee: CARGILL, INCORPORATED, Wayzata, MN (US)

(21) Appl. No.: 18/705,792

(22) PCT Filed: Oct. 4, 2022
(86) PCT No.: PCT/US2022/077526

§ 371 Date: Apr. 29, 2024

Related U.S. Application Data
(60) Provisional application No. 63/263,848, filed on Nov. 10, 2021.

(51) Int. Cl. A61K 8/73; A61K 8/9717; A61Q 15/00

(52) U.S. Cl. CPC A61K 8/732; A61K 8/9717; A61Q 15/00; A61K 2800/52

(57) ABSTRACT

The present technology provides a natural-based deodorant composition that includes about 5.0 weight percent (wt %) to about 15.0 wt % of a texturizer based on total weight of the deodorant composition, about 0.5 wt % to about 2.5 wt % of a gelling agent comprising kappa carrageenan; and about 5.0 wt % to about 35.0 wt % of an emollient composition; and water; wherein the texturizer includes a n-octenyl succinate anhydride (nOSA) modified starch and a hydroxypropylated starch, wherein a weight ratio of the nOSA modified starch to hydroxypropylated starch is about 3:1 to about 20:1.

### CROSS-REFERENCE TO RELATED APPLICATIONS

This application claims the benefit of U.S. Provisional Patent Application No. 63/263,848, filed Nov. 10, 2021, which is incorporated by reference herein in its entirety.

### TECHNICAL FIELD

The present technology relates to natural-based deodorant compositions.

### BACKGROUND

Deodorants are cosmetic products used to mask or reduce body odor, particularly, offensive body odor. Fragrances and natural oils are commonly used ingredients for masking capability. Additionally, bactericidal/bacteriostatic agents such as triclosan, zinc oxide or salicylic acid can be used in addition to fragrances/natural oils.

Deodorants and antiperspirants are used interchangeably as they both fall in an area related to sweat production and its associated odor. However, deodorants and antiperspirants work by different mechanisms. Sweat as secreted and produced by sweat (eccrine) glands is odorless. However, sweat generates an odor when it undergoes bacterial decomposition. While deodorants work to mask body/sweat odor, antiperspirants are products that work to block or reduce the amount of sweat produced. It is important to note that while some antiperspirants can have deodorizing capacity, pure deodorants do not have antiperspirant like activity. Typically, deodorants are classified as “cosmetics” whereas, antiperspirants are classified as “over the counter” products by the United States Food and Drug Administration (FDA).

There are many commercial deodorant products available in the market. In addition, superior sensory texture is still in demand. Accordingly, with ever-growing trends towards environmentally friendly cosmetic products, consumers increasingly demand deodorants with more or only naturally based, sustainable and biodegradable ingredients. Thus, it would be advantageous to have improved natural-based deodorant compositions.

### SUMMARY

In one aspect, the present technology provides a natural-based deodorant composition that includes a texturizer composition containing a n-octenyl succinic anhydride modified starch, a hydroxypropylated starch, or combinations thereof: a gelling agent that includes kappa carrageenan, and an emollient composition. Preferably, as described in any aspect herein, the natural-based deodorant includes about 5.0 weight percent (wt %) to about 15.0 wt % of a texturizer based on total weight of the deodorant composition; about 0.5 wt % to about 2.5 wt % of a gelling agent that includes kappa carrageenan; about 5.0 wt % to about 35.0 wt % of an emollient composition; and water; wherein the texturizer includes a n-octenyl succinate anhydride (nOSA) modified starch and a hydroxypropylated starch, wherein a weight ratio of the nOSA modified starch to hydroxypropylated starch is about 3:1 to about 20:1.

In another aspect, the present technology provides a method of stabilizing a cosmetic deodorant composition. The method includes preparing a first mixture comprising a gelling agent that includes kappa carrageenan and water; contacting the first mixture with a texturizer; preparing a second mixture that includes an emollient composition, wherein the emollient composition includes an edible solid fat and an edible liquid fat; heating the first mixture and the second mixture to a temperature of about 65° C. (149° F.) to about 85° C. (185° F.); and combining the first mixture and the second mixture to obtain a stabilized cosmetic deodorant; wherein the texturizer includes a n-octenyl succinate anhydride (nOSA) modified starch and a hydroxypropylated starch, wherein a weight ratio of the nOSA modified starch to hydroxypropylated starch is about 3:1 to about 20:1.

In another aspect, the present technology provides a natural-based deodorant prepared according to the method as described herein.

In yet another aspect, the present technology provides the use of a texturizer that includes a n-octenyl succinic anhydride modified starch and a hydroxypropyl starch, a gelling agent that includes kappa carrageenan, and a thickener to stabilize a cosmetic deodorant. Preferably, the cosmetic deodorant is a natural-based deodorant.

### BRIEF DESCRIPTION OF THE DRAWINGS

FIG. 1 illustrates a graph of the cone penetration values measured for commercial deodorant samples, comparative natural-based deodorant samples (Formulations 3-8) and natural-based deodorant samples according to one or more aspects of the present technology (Formulations 1B, 1E, 1F, 1G, and 1I).

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter. One aspect described in conjunction with a particular aspect is not necessarily limited to that aspect and can be practiced with any other aspect(s).

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, the singular forms “a,” “an,” and “the” and similar referents in the context of describing the elements (especially in the context of the following claims) include plural referents unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context.

Nothing in the cited references teaches or suggests the claimed process. In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section.

As used herein, the terms “for example,” “for instance,” “such as,” or “including” are meant to introduce examples that further clarify more general subject matter. Unless otherwise specified, these examples are provided only as an aid for understanding the applications illustrated in the present disclosure, and are not meant to be limiting in any fashion.

In the methods described herein, the acts can be carried out in a specific order as recited herein. Alternatively, in any aspect disclosed herein, specific acts may be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, plus or minus within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

As used herein, the following terms have the following meanings unless expressly stated to the contrary.

The term “natural-based” as used herein refers to a composition in which the formulation primarily includes ingredients derived from plant or animal sources.

The term “carrageenan” as used herein refers to a family of poly disperse long chain galactans, which can be extracted from seaweed (e.g., red seaweeds). The basic structure of carrageenans is a linear polysaccharide made up of a repeating disaccharide sequence of β-D-galactopyranose linked through positions 1,3 (A residues) and α-D-galactopyranose residues linked through positions 1,4 (B residues). The regular backbone structure of the basic carrageenans is disrupted by a more or less ordered distribution of sulphate groups. Some of the galactose units have attached sulfate groups while others are unsulfated. The three main types (iota, kappa, and lambda) of carrageenan molecules differ by (1) the types of linkages between the galactose units, and (2) the point of attachment of the sulfate groups to the galactose units. These apparently small differences in chemical constitution and structure make major differences in the properties of each type of carrageenan.

Gelling in carrageenan is caused by helix formation and this can only occur in repeat structures where the B residue is in a 1-C-4 conformation. Lambda carrageenan (theoretically having 3 sulphate groups per repeating unit) has both its sugar residues in a 4-C-1 conformation and therefore is not typically active in gel formation. The gelling types of carrageenan include the kappa type (theoretically having 1 sulphate group per repeating unit) and the iota type (theoretically having 2 sulphate groups per repeating unit): all containing a 3,6-anhydro bridge on the B unit which forces the sugar to flip from a 4-C-1 conformation to a 1-C-4 conformation, allowing for cross-linking and gel formation.

A “triacylglyceride” refers to a molecule having a glycerol moiety that is linked to three fatty acid residues via ester bonds. The terms “triacylglycerol,” “triacylglyceride,” “triglyceride,” and “TAG” are used interchangeably herein.

The term “fatty acid” as used herein can refer to a molecule comprising a hydrocarbon chain and a terminal carboxylic acid group. As used herein, the carboxylic acid group of the fatty acid may be modified or esterified, for example as occurs when the fatty acid is incorporated into a glyceride or another molecule (e.g., COOR, where R refers to, for example, a carbon atom). Alternatively, the carboxylic acid group may be in the free fatty acid or salt form (i.e., COO″ or COOH). The ‘tail’ or hydrocarbon chain of a fatty acid may also be referred to as a fatty acid chain, fatty acid sidechain, or fatty chain. The hydrocarbon chain of a fatty acid will typically be a saturated or unsaturated aliphatic group. A fatty acid having N number of carbons, will typically have a fatty acid side chain having N−1 carbons. However, the subject application also relates to modified forms of fatty acids, e.g., epoxidized fatty acids, and thus the term fatty acid may be used in a context in which the fatty acid has been substituted or otherwise modified as described.

A “fatty acid residue” is a fatty acid in its acyl or esterified form.

A “saturated” fatty acid is a fatty acid that does not contain any carbon-carbon double bonds in the hydrocarbon chain. An “unsaturated” fatty acid contains one or more carbon-carbon double bonds. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted cis and trans. Naturally occurring unsaturated fatty acids are generally in the “cis” form. Epoxidized renewable oil or fat may include one or more epoxide rings formed from cis or trans carbon-carbon double bonds.

Non-limiting examples of fatty acids include C8, C10, C12, C14, C16 (e.g., C16:0, C16:1), C18 (e.g., C18:0, C18:1, C18:2, C18:3, C18:4), C20 and C22 fatty acids. For example, the fatty acids can be caprylic (8:0), capric (10:0), lauric (12:0), myristic (14:0), palmitic (16:0), stearic (18:0), oleic (18:1), linoleic (18:2) and linolenic (18:3) acids.

As used herein, the term “humectant” refers to a wetting or moistening agent, which aids in maintaining or retention of moisture in a product or formulation.

As used herein, the term “starch” refers to a polymeric carbohydrate consisting of a large number of glucose units joined by glycosidic bonds. This polysaccharide is produced by most green plants as energy storage. It is the most common carbohydrate in human diets and is contained in large amounts in staple foods. Suitable starches for use in the present technology may come from any plant source including but not limited to: potatoes, wheat, maize (corn), rice, tapioca, quinoa, cassava, and the like. Pure starch is a white, tasteless and odorless powder that is insoluble in cold water or alcohol. It consists of two types of molecules: the linear and helical amylose and the branched amylopectin. Depending on the plant, starch generally contains 1 to 25% amylose and 75 to 99% amylopectin by weight. When it is isolated directly from the plant source it is most often referred to as “native starch”. Native starch requires heat to thicken or gelatinize. When a starch is pre-cooked, it can then be used to thicken instantly in cold water. This is referred to as a pregelatinized starch.

Native starch can be hydrolyzed into simpler carbohydrates by temperature, acids, various enzymes, or a combination of the three. The resulting fragments are known as dextrins or hydrolyzed starch. The extent of conversion is typically quantified by dextrose equivalent (DE), which is roughly the fraction of the glycosidic bonds in starch that have been broken. For example, maltodextrin is a lightly hydrolyzed (DE 4-20 or 10-20) starch product used as a bland-tasting filler and thickener. Various glucose syrups (DE 30-70), also called com syrups in the US, are a type of hydrolyzed starch that are viscous solutions used as sweeteners and thickeners in many kinds of processed foods. Dextrose (DE 100), commercial glucose, is prepared by the complete hydrolysis of starch.

The starch may also be a cyclodextrin. Cyclodextrins are a well-known family of cyclic oligosaccharides, consisting of a macrocyclic ring of glucose subunits joined by a-1,4 glycosidic bonds. Cyclodextrins are produced from starch by enzymatic conversion. They are used in food, pharmaceutical, drug delivery, and chemical industries, as well as agriculture and environmental engineering. Cyclodextrins are composed of 5 or more α-D-glucopyranoside units linked l→4, as in amylose (a fragment of starch). The largest cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape including: 1) a (alpha)-cyclodextrin: 6 glucose subunits: 2) b (beta)-cyclodextrin: 7 glucose subunits; and 3) g (gamma)-cyclodextrin: 8 glucose subunits.

The present technology provides natural-based deodorant compositions. The deodorant composition as described herein advantageously provides a minimalist approach to the INCI (International Nomenclature Cosmetic Ingredient) list of ingredients, where the present technology provides a clean and simple cosmetic deodorant formulation which surprisingly exhibits good functional properties of easy spreading, non-sticky/tacky skin feel, good pick-up, smooth application, and excellent processability.

Deodorants are expected to not only mask odor, but deodorants are also expected to have other properties important to consumers such as ease of use, quick drying, and soft and smooth application of product giving superior sensory. Typically, consumer acceptance is evaluated based on a non-sticky, no-residue product which is pleasant and easy to use.

Deodorants come in various application forms. Typically, such forms may include emulsions, gel or solid sticks, roll-ons, sprays, and even deodorant soaps. A typical deodorant formula contains hydrophilic solvent (mostly glycols), water, gelling agent (mostly sodium stearate but sodium palmitate, sodium arachidate and sodium behenate can be utilized) antioxidant, antimicrobial agent, chelating agent, fragrance, colorant and others. Deodorant creams/lotions are oil in water emulsions that contain fatty alcohol, ethoxylated fatty alcohols as texturizers. Typically, deodorant sticks may contain oils, fatty alcohols, and functional silicones elastomers. Commonly used ingredients as ‘active ingredients’ in deodorants are triclosan, salicylic acid, triclocarban, quaternary ammonium compounds, metal salts like aluminum and zirconium salts, glycols.

Typically, deodorants should have a good pay-off so that the product when applied onto skin will deposit a thin layer on the skin, along with easy/good spreadability. In addition, typical deodorant products should also have a non-transfer property so that after application on skin it will not transfer on and/or avoid staining clothes.

The present inventors have formulated a cosmetic deodorant stick to meet consumer demand and create a formulation containing nature-derived ingredients. The present inventors employing the minimalist approach of the INCI list of ingredients to provide a clean and simple natural-based deodorant formulations giving good functional properties of easy spreading, a non-sticky/tacky skin feel and smooth application of product.

The inventors surprisingly discovered that a combination of a starch texturizer system (e.g., an anhydride modified starch and hydroxypropylene starch) and carrageenan (e.g., kappa carrageenan) provide natural-based deodorant formulations exhibiting improved functional properties (i.e., good pick-up, improved spreadability, non-sticky/tacky skin feel, smooth application, and structural hardness).

In one aspect, the present technology provides a natural-based deodorant composition that includes a texturizer composition containing an anhydride modified starch, a hydroxypropylated starch, or combinations thereof: a gelling agent that includes kappa carrageenan, and an emollient composition.

Preferably, the natural-based deodorant composition includes about 5.0 weight percent (wt %) to about 15.0 wt % of a texturizer based on total weight of the deodorant composition; about 0.5 wt % to about 2.5 wt % of a gelling agent that includes kappa carrageenan; about 5.0 wt % to about 35.0 wt % of an emollient composition; and water; wherein the texturizer includes a n-octenyl succinate anhydride (nOSA) modified starch and a hydroxypropylated starch, wherein a weight ratio of the nOSA modified starch to hydroxypropylated starch is about 3:1 to about 20:1.

The deodorant may include about 5.0 wt % to about 15.0 wt %, about 5.5 to about 12.5 wt %, about 6.0 wt % to about 11.0 wt %, about 7.0 wt % to about 10.0 wt % of the texturizer, based on total weight of the deodorant composition, or any value including and/or in between any two of the preceding values. Suitably amounts of the texturizer may include about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, about 10.0 wt %, about 10.5 wt %, about 11.0 wt %, about 11.5 wt %, about 12.0 wt %, about 12.5 wt %, about 13.0 wt %, about 13.5 wt %, about 14.0 wt %, about 14.5 wt %, about 15.0 wt %, or any range including and/or in between any two of the preceding values.

As described herein, a “modified starch” refers to a starch that has been altered from its native state, resulting in modification of one or more of its chemical or physical properties. Starches may be modified, for example, by enzymes, oxidation or, substitution with various compounds. For example, starches can be modified to increase stability against heat, acids, or freezing, improve texture, increase or decrease solubility, among others. Modified starches may be partially or completely degraded into shorter chains or glucose molecules. Amylopectin may be debranched. In one example, modified starches are cross-linked for example to improve stability. Starches that are modified by substitution have a different chemical composition. For example, starches may be modified with an anhydride of a polyacid, said anhydride being preferably chosen from the group that includes maleic and succinic anhydrides, more preferably from the group that includes (alkyl-, alkenyl-, aralkyl- or aralkenyl-) succinic anhydrides. More preferably the succinic anhydride has a chain length between 3 and 22 carbons or specifically a chain length of 8, 10, 12, 14, 16, 18 or combinations thereof. As described herein in any aspect, said anhydride is n-octenyl succinic anhydride (nOSA). Methods of making said anhydride modified starches are generally described in PCT Application Serial No. PCT/US2020/050110 entitled “MODIFIED STARCHES FOR HOME CARE AND PERSONAL CARE,” which are hereby incorporated by reference herein in its entirety.

The nOSA modified starch is a modified starch that has been partially substituted by reaction with n-octenyl succinic anhydride. nOSA starches can also be characterized by the degree of substitution of the n-octenyl succinic groups. The degree of substitution is abbreviated as “DS”. It is a well understood and used nomenclature by those of skill in the art. For example, food grade approved nOSA starches have a DS of 0.001 to 0.03. This means that about 0.1%-3.0% of potential loci have been substituted by n-octenyl succinic groups. Typically, the maximum allowable DS for nOSA starch for food use is 0.03 or 3.0% substitution. Preferably, the nOSA modified starches may include C*EmTex 12688 (sodium starch octenylsuccinate) commercially available from Cargill, Incorporated.

The texturizer also includes a hydroxypropylated starch. Hydroxypropylated starch is another example of a modified starch that has been functionalized by hydroxypropylation. Such hydroxypropylated starches are well known in the art and are “E-coded” under the designation 1400 in the International System for Food Additives (INS). Preferred hydroxypropylated starches of the present invention include C*HiForm 12748 commercially available from Cargill, Incorporated. In any aspect herein, the hydroxypropylated starch may be hydroxypropyl starch phosphate.

The texturizer has a weight ratio of the nOSA modified starch to the hydroxypropylated starch of about 3:1 to about 20:1, preferably about 3:1 to about 10:1. Suitable weight ratios may include, but are not limited to, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, or any range including and/or in between any two of the preceding values.

The texturizer may be a blend of the nOSA modified starch and the hydroxypropylated starch. As used herein, the term “blend” refers to a physical mixture of two or more substances. For example, the texturizer may include a blend of sodium starch octenylsuccinate and hydroxypropyl starch phosphate. In any aspect described herein, the texturizer may include a blend, wherein the nOSA modified starch is present in an amount of about 75 wt % to about 95 wt %, or preferably about 75 wt % to about 85 wt %; suitable amounts of the nOSA modified starch in the blend may be about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, or any range including and/or in between any two of the preceding values. In any aspect described herein, the texturizer may include a blend, wherein the hydroxypropylated starch is present in an amount of about 5 wt % to about 25 wt %.

The deodorant composition may include about 0.5 wt % to about 2.5 wt %, about 1.0 wt % to about 2.3 wt %, or about 1.2 wt % to about 1.8 wt % of the gelling agent based on total weight of the deodorant composition. For example, suitable amounts of the gelling agent may include about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, or any range including and/or in between any two of the preceding values. Preferably the gelling agent may include only kappa carrageenan. Additionally or alternatively, the gelling agent may further include one or more additional gelling agents, including but not limited to, iota carrageenan, schleroglucan, or mixtures thereof.

The deodorant composition includes an emollient, wherein the emollient composition includes an edible solid fat, an edible liquid fat, or combinations thereof. As used herein, the terms “edible solid fat”, “edible liquid fat”, or “edible oil” refer to a fat or oil that is suitable for human consumption. Edible solid fats, edible liquid fats, and edible oils are typically compositions including triacylglycerols (“TAG”). The edible solid fats, edible liquid fats, and edible oils may be obtained from plant, animal, or microbial sources.

Generally, an edible solid fat that is in a solid or semi-solid state at room temperature (i.e., approximately about 20° C. to about 25° C.) and ambient pressure (i.e., approximately 1 atm), an edible liquid fat that is in a liquid or semi-liquid state at room temperature and ambient pressure having a SFC of at least about 1.0 wt %, or mixtures thereof. Edible solid or liquid fats, as described herein, may be natural fats (i.e., unmodified), modified fats, or mixtures thereof. For example, the modified fat may be a hydrogenated fat, a chemically or enzymatically interesterified fat, a fractionated fat, or mixtures thereof.

Suitable edible solid fats may include, but are not limited to, butter, lard, tallow, butter oil, cocoa butter, mango butter, shea butter, milk fat, coconut oil, palm oil, palm olein, palm kernel oil, shea oil, illipe oil, sal oil, kokum gurgi oil, mango kernel oil, hydrogenated vegetable oil (such as hydrogenated coconut oil, hydrogenated fractionated palm kernel oil, hydrogenated cottonseed oil, hydrogenated soy bean oil, hydrogenated sunflower oil, hydrogenated canola oil, hydrogenated rapeseed oil, and the like or mixtures thereof), hydrogenated fish oil, or mixtures of two or more thereof. As used herein, “vegetable oils” refers to oils derived from vegetables and/or oil seeds. Typically, the edible solid fat may be cocoa butter, hydrogenated vegetable oil, or mixtures of two or more thereof. Preferably, the edible solid fat may be selected from hydrogenated coconut oil, cocoa butter, or mixtures thereof.

Suitable edible liquid fats and/or oils are typically in a liquid state at room temperature (i.e., approximately about 20° C. to about 25° C.) and ambient pressure (i.e., approximately 1 atm). In some aspects, the edible liquid fats and/or oils are free, or substantially free, of crystallized solid fat. The edible liquid fats, as described herein, may be natural oils (i.e., unmodified), modified oils, or mixtures thereof. For example, the edible oil may be a hydrogenated oil, a chemically or enzymatically interesterified oil, a fractionated oil, or mixtures thereof. The edible liquid fats may include, but are not limited to, sunflower oil, citrus oil (such as lemon oil, orange oil, and the like or mixtures thereof), grape seed oil, sesame oil, peanut oil, mustard oil, nut oil (such as almond oil, cashew oil, walnut oil, hazelnut oil, macadamia oil, or mixtures thereof), corn oil, wheat kernel oil, rapeseed oil, safflower oil, flaxseed oil, soy bean oil, canola oil, cottonseed oil, marine oil (such as fish oil, algal oil, fungal oil, or mixtures thereof), rice bran oil, olive oil, or mixtures of two or more thereof. The edible liquid fats may be a high oleic edible oil, such as high oleic sunflower oil, high oleic citrus oil, high oleic grape seed oil, high oleic sesame oil, high oleic peanut oil, high oleic mustard oil, high oleic nut oil, high oleic corn oil, high oleic wheat kernel oil, high oleic rapeseed oil, high oleic safflower oil, high oleic flaxseed oil, high oleic soy bean oil, high oleic canola oil, high oleic cottonseed oil, high oleic marine oil, or mixtures of two or more thereof. Preferably, the edible liquid fats are high oleic sunflower oil.

The emollient composition may have a weight ratio of the edible solid fat to the edible liquid fat of about 1:1 to about 1:3, and preferably of about 1:1.3 to about 1:2. For example, the weight ratio of the edible solid fat to the edible liquid fat in the emollient composition may be about 1:1, about 1:1.1, about 1:1.3, about 1:1.5, about 1:1.7, about 1:1.9, about 1:2, about 1:2.1, about 1:2.3, about 1:2.5, about 1:2.7, about 1:2.9, about 1:3, or any range including and/or in between any two of the preceding values.

The deodorant includes about 5.0 wt % to about 35.0 wt % of an emollient composition based on total weight of the deodorant composition, preferably about 15.0 wt % to about 30.0 wt %, and most preferably, about 20.0 wt % to about 30.0 wt %. For example, suitable amounts of the emollient composition may include about 5.0 wt %, about 6.0 wt %, about 7.0 wt %, about 8.0 wt %, about 9.0 wt %, about 10.0 wt %, about 11.0 wt %, about 12.0 wt %, about 13.0 wt %, about 14.0 wt %, about 15.0 wt %, about 16.0 wt %, about 17.0 wt %, about 18.0 wt %, about 19.0 wt %, about 20.0 wt %, about 21.0 wt %, about 22.0 wt %, about 23.0 wt %, about 24.0 wt %, about 25.0 wt %, about 26.0 wt %, about 27.0 wt %, about 28.0 wt %, about 29.0 wt %, about 30.0 wt %, about 31.0 wt %, about 32.0 wt %, about 33.0 wt %, about 34.0 wt %, about 35.0 wt %, or any range including and/or in between any two of the preceding values.

Water is present to make up the balance of the deodorant composition. While any amount of water may include in the deodorant composition to make up the balance, typically water may be present in the deodorant composition in an amount of about 5.0 wt % to about 75.0 wt % based on total weight of the deodorant composition. For example, the amount of water present in the deodorant composition is about 5.0 wt %, about 10.0 wt %, about 15.0 wt %, about 20.0 wt %, about 25.0 wt %, about 30.0 wt %, about 35.0 wt %, about 40.0 wt %, about 45.0 wt %, about 50.0 wt %, about 55.0 wt %, about 60.0 wt %, about 65.0 wt %, about 70.0 wt %, about 75.0 wt %, or any range including and/or in between any two of the preceding values. Preferably the deodorant composition may include water in an amount of about 35.0 wt % to about 60.0 wt %, about 40.0 wt % to about 55.0 wt %, or about 45.0 wt % to about 50.0 wt %.

The deodorant composition as described herein may further include one or more additives that may be selected from thickeners, humectants, antioxidants, antimicrobial agents, chelating agents, colorants, fragrances, additional emollients, conditioning agents, butters, waxes, starches, gums, clays, essential oils, plant extracts, odor absorbers, or combinations thereof.

The deodorant composition may further include sodium stearate, lithium stearate, aluminum stearate, aluminum distearate, aluminum tristearate, calcium stearate, magnesium stearate, potassium stearate, zinc stearate, or combinations thereof. The one or more thickeners may be present in the deodorant composition in amounts of about 1.0 wt % to about 25.0 wt % based on total weight of the deodorant composition. Suitable amounts of the one or more thickeners may include about 1.0 wt %, about 2.0 wt %, about 3.0 wt %, about 4.0 wt %, about 5.0 wt %, about 6.0 wt %, about 7.0 wt %, about 8.0 wt %, about 9.0 wt %, about 10.0 wt %, about 11.0 wt %, about 12.0 wt %, about 13.0 wt %, about 14.0 wt %, about 15.0 wt %, about 16.0 wt %, about 17.0 wt %, about 18.0 wt %, about 19.0 wt %, about 20.0 wt %, about 21.0 wt %, about 22.0 wt %, about 23.0 wt %, about 24.0 wt %, about 25.0 wt %, or any range including and/or in between any two of the preceding values.

The deodorant composition may further include one or more humectants selected from glycerin, sorbitol, propylene glycol, triethylene glycol, tripropylene glycol, polypropylene glycol, butylene glycol, urea, aloe vera juice, and combinations thereof. The one or more humectants may be present in the deodorant composition in amounts of about 5.0 wt % to about 25.0 wt % based on total weight of the deodorant composition. Suitable amounts of the one or more humectants may include about 5.0 wt %, about 6.0 wt %, about 7.0 wt %, about 8.0 wt %, about 9.0 wt %, about 10.0 wt %, about 11.0 wt %, about 12.0 wt %, about 13.0 wt %, about 14.0 wt %, about 15.0 wt %, about 16.0 wt %, about 17.0 wt %, about 18.0 wt %, about 19.0 wt %, about 20.0 wt %, about 21.0 wt %, about 22.0 wt %, about 23.0 wt %, about 24.0 wt %, about 25.0 wt %, or any range including and/or in between any two of the preceding values.

The deodorant composition may further include one or more antioxidants selected from vitamin E, vitamin A, vitamin C, butylated hydroxytoluene (BHT), butylated (BHA), natural plant extracts, and combinations thereof. The one or more antioxidants may be present in the deodorant composition in amounts of about 0 wt % to about 1.5 wt % based on total weight of the deodorant composition. Suitable amounts of the one or more antioxidants may include about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, or any range including and/or in between any two of the preceding values.

The deodorant composition may further include one or more chelating agents selected from the group consisting of ethylene diamine tetraacetic acid (EDTA), citric acid, sodium phytate, gluconodelta lactone, or combinations thereof. The one or more chelating agents may be present in the deodorant composition in amounts of about 0 wt % to about 0.5 wt % based on total weight of the deodorant composition. Suitable amounts of the one or more chelating agents may include about 0.01 wt %, about 0.05 wt %, about 0.1 wt %, about 0.2 wt %, about 0.3 wt %, about 0.4 wt %, about 0.5 wt %, or any range including and/or in between any two of the preceding values.

The deodorant composition as described herein in any aspect can be in a solid form. As such, the deodorant composition as described herein exhibits good structural hardness such that the deodorant maintains a desirable stick shape. In any aspect disclosed herein, the deodorant composition may have a cone penetration of about 90 mm to about 140 mm, preferably a cone penetration of about 95 mm to about 130 mm. For example, the deodorant may have a cone penetration of about 90 mm, about 95 mm, about 100 mm, about 105 mm, about 110 mm, about 115 mm, about 120 mm, about 125 mm, about 130 mm, about 135 mm, about 140 mm, or any range including and/or in between any two of the preceding values. In any aspect, the deodorant composition may have a cone penetration of about 90 mm to about 140) mm as measured according to ASTM D 937-97 (Reapproved 2002). In certain aspects, the deodorant composition as described herein can be formulated as a solid deodorant stick.

The deodorant composition may further include one or more active agents as described herein. For example, the active agent may be zinc oxide, sodium bicarbonate, or combinations thereof. Preferably, the active agent may include zinc oxide.

The deodorant as described herein typically exhibits one or more improved functional properties, including but not limited to, excellent processability, good pay-off, easy spreadability, improved skin feel (such as improved non-sticky, non-tacky skin feel), or combinations of two or more thereof.

In another aspect, the present technology provides a method of stabilizing a cosmetic deodorant composition. The method includes preparing a first mixture comprising a gelling agent that includes kappa carrageenan and water; contacting the first mixture with a texturizer; preparing a second mixture that includes an emollient composition, wherein the emollient composition includes an edible solid fat and an edible liquid fat; heating the first mixture and the second mixture to a temperature of about 65° C. to about 85° C.; and combining the first mixture and the second mixture to obtain a stabilized cosmetic deodorant; wherein the texturizer includes an anhydride modified starch prepared from a base starch having an average molecular weight of about 15,000 to about 200,000 g/mol and a degree of substitution of about 10% to about 80%; and a hydroxypropylated starch.

The first mixture may further include one or more additives as described in the present disclosure, wherein the one or more additives may be selected from thickeners, humectants, antioxidants, antimicrobial agents, chelating agents, colorants, fragrances, additional emollients, conditioning agents, butters, waxes, starches, gums, clays, essential oils, plant extracts, odor absorbers, or combinations thereof

The first mixture may further include an active agent selected from zinc oxide, sodium bicarbonate, or mixtures thereof. Preferably, the active agent may be zinc oxide (ZnO).

Preferably the cosmetic deodorant prepared according to the method of the present technology is a natural-based deodorant as described herein in any aspect.

The cosmetic deodorant prepared according to the method of the present technology is in a solid form and exhibits a cone penetration of about 90 mm to about 140 mm, preferably a cone penetration of about 95 mm to about 130 mm. For example, the cosmetic deodorant may have a cone penetration of about 90 mm, about 95 mm, about 100 mm, about 105 mm, about 110 mm, about 115 mm, about 120 mm, about 125 mm, about 130 mm, about 135 mm, about 140 mm, or any range including and/or in between any two of the preceding values. In any aspect, the cosmetic deodorant composition may have a cone penetration of about 90 mm to about 140 mm as measured according to ASTM D 937-97 (Reapproved 2002). In certain aspects, the cosmetic deodorant prepared according to the present method is formulated as a solid deodorant stick.

The cosmetic deodorant prepared according to the method of the present technology exhibits one or more improved functional properties that includes excellent processability, good pay-off, easy spreadability, improved skin feel, or combinations thereof.

In another aspect, the present technology provides a natural-based deodorant prepared according to the method as described herein.

In another aspect, the present technology provides the use of a texturizer that includes an anhydride modified starch and hydroxypropyl starch, a gelling agent that includes kappa carrageenan, and a thickener to stabilize a cosmetic deodorant. Preferably, the cosmetic deodorant is a natural-based deodorant.

The present invention, thus generally described, will be understood more readily by reference to the following examples, which are provided by way of illustration and are not intended to be limiting of the present invention.

### EXAMPLES

TABLE 1
Ingredients

| Phase | Component | Material | INCI | Trade Name | Supplier |
|---|---|---|---|---|---|
| A | Water | | Water (aqua) | — | — |
| A | Gelling | Kappa | Carrageenan | Satiagel VPC 614 | Cargill |
| | Agent | Carrageenan | | | |
| B | Humectant | Glycerin | Glycerin | Glycerin | Cargill |
| B | Active | Zinc Oxide | Zinc Oxide (and) | Zinc Oxide | |
| | Agent | | Caprylic/Capric | (GC70MZCJ-G) | |
| | | | Triglyceride (and) | | |
| | | | Jojoba Esters (and) | | |
| | | | Glyceryl | | |
| | | | Behenate/Eicosadioate | | |
| C | Antioxidant | EDTA | Tetrasodium 60 | EDTA BX Pulver | |
| | | | EDTA | | |
| C | Thickener | Sodium | Sodium Stearate | Sodium Stearate | |
| | | Stearate | | OP-200V | |
| D | Texturizer | Starch | Sodium Starch | StarDesign ™ | Cargill |
| | | | Octenylsuccinate | Power | |
| | | | (and) hydroxypropyl | | |
| | | | starch phosphate | | |
| E | Emollient | Hydrogenated | Hydrogenated | RBD HYD 92 | Cargill |
| | | Coconut Oil | Coconut Oil | COC Oil KP Blk | |
| E | Emollient | Cocoa Butter | Theobroma Cacao | Refined White | Cargill |
| | | | (Cocoa) Seed Butter | Cocoa Butter | |
| E | Oil | High Oleic | Helianthus Annuus | High Oleic | Cargill |
| | | Sunflower Oil | Seed Oil | Sunflower Oil | |
| | | | | AP80 | |
| E | Antioxidant | Vitamin E | D-Alpha Tocopheryl | Vitamin E | Cargill |
| | | | Acetate | | |

### Example 1: Preparation of Natural-Based Deodorant with Zinc Oxide Active

Natural-based deodorants containing zinc oxide active agent were prepared by combining the ingredients described in Table 1. Water was added to an appropriately sized beaker and secured on a blender stand and hot plate. The blender rod was lowered into the water and above beaker glass bottom. While stirring at 500 rpm, 1.5 wt % carrageenan was added and allowed to blend completely with continuous heating at 60° C. to form phase A. Glycerin and zinc oxide were measured out and combined in a separate container to form phase B. Once phase A was blended and maintained at a temperature 60° C., phase B was added to phase A and heated to a temperature of 70° C.

Sodium Stearate and EDTA were measured out and combined in a separate container to obtain phase C. Phase C was added to the combined phases A and B and maintained at a temperature of 70° C. Once the combined phases A, B, and C were blended completely, StarDesign™ Power (phase D) was added to the combined phases A, B, and C. The mixture was allowed to blend and heated to 75° C. All the ingredients of phase E (Table 1) were added to a separate beaker and heated on a hot plate to a temperature of 75° C. The mixture of phases A, B, C, and D was heated to a temperature of 75° C., and phase E was added slowly to the mixture of phases A, B, C, and D. The combined phases were blended until completely mixed, and the mixture was poured into a container to cool to obtain the natural-based deodorant.

Natural-based deodorant formulations provided in Table 2 were prepared according to the above procedure.

TABLE 2
Natural-based deodorants with ZnO active

| | Formulation (% conc.) | | | | | | | | |
|---|---|---|---|---|---|---|---|---|---|
| | 1A | 1B | 1C | 1D | 1E | 1F | 1G | 1H | 1I |
| | [[16]] | [[16U]] | [16V]] | [[16W]] | [[16X]] | [16Y]] | [[16Z]] | [16A1]] | [[16B1]] |
| Ingredients (wt %) | | | | | | | | | |
| Water | 49.3 | 47.8 | 47.55 | 47.05 | 46.8 | 49.3 | 48.3 | 46.3 | 45.3 |
| Satiagel ™ VPC 614 | 1.5 | 1.0 | 1.25 | 1.75 | 2.0 | 1.5 | 1.5 | 1.5 | 1.5 |
| Tetrasodium EDTA | 0.1 | 0.1 | 0.1 | 0.1 | 0.1 | 0.1 | 0.1 | 0.1 | 0.1 |
| Sodium Stearate | 5.0 | 5.0 | 5.0 | 5.0 | 5.0 | 5.0 | 5.0 | 5.0 | 5.0 |
| Glycerin | 10.0 | 10.0 | 10.0 | 10.0 | 10.0 | 10.0 | 10.0 | 10.0 | 10.0 |
| ZnO | 0 | 2.0 | 2.0 | 2.0 | 2.0 | 2.0 | 2.0 | 2.0 | 2.0 |
| StarDesign ™ Power | 8.0 | 8.0 | 8.0 | 8.0 | 8.0 | 6.0 | 7.0 | 9.0 | 10.0 |
| High Oleic | 15.0 | 15.0 | 15.0 | 15.0 | 15.0 | 15.0 | 15.0 | 15.0 | 15.0 |
| Sunflower oil | | | | | | | | | |
| Hydrogenated | 3.0 | 3.0 | 3.0 | 3.0 | 3.0 | 3.0 | 3.0 | 3.0 | 3.0 |
| Coconut oil | | | | | | | | | |
| White Cocoa | 8.0 | 8.0 | 8.0 | 8.0 | 8.0 | 8.0 | 8.0 | 8.0 | 8.0 |
| Butter | | | | | | | | | |
| Vitamin E | 0.1 | 0.1 | 0.1 | 0.1 | 0.1 | 0.1 | 0.1 | 0.1 | 0.1 |

Formulation 1A represents a base formulation of the natural-based deodorant, which contains no active agent (e.g., ZnO). Formulation 1A exhibited the desired properties of good pay-off and the balance of maintaining good strength and structure without diminishing the pay-off property. The desired pay-off was such that a good film can be obtained when the product is applied/rubbed on the skin. Formulation 1A exhibited excellent processing, structure forming, product application, and sensory feel.

Formulations 1B-1J contain 2.0 wt % of ZnO as the active agent for absorption of excess sebum and preventing odor. Formulations 1B-1E evaluated the impact of varying the concentration of kappa carrageenan in combination with ZnO active in the natural-based deodorant to meet the desired functional and sensorial benefits. As shown in Table 2, kappa carrageenan concentration was varied from 1 wt %, 1.25 wt %, 1.5 wt %, 1.75 wt %, and 2 wt %.

At levels of 1 wt % kappa carrageenan (formulation 1B), the formulation was softer than the base formulation and exhibited a higher pay-off. Formulations 1C and 1D containing 1.25 wt % and 1.75 wt % of kappa carrageenan, respectively, exhibited excellent processability and sensory feel on application when compared to formulation 1A. Formulation 1E containing 2 wt % kappa carrageenan exhibited a crumbly texture when compared to formulations 1A-1D.

Formulations 1F-1I evaluated the benefits of the natural-based deodorant containing 2 wt % ZnO active, where the texturizer concentration (i.e., StarDesign™ Power) is varied from 6 wt % to 10 wt %. Formulation 1F exhibited some water leaching, indicative of some loss of stability over time. Formulations 1G-1I exhibited excellent processing, structural hardness, and functional needs (e.g., good pay-off).

### Example 2: Natural-Based Deodorants with Sodium Bicarbonate Active

Natural-based deodorants containing sodium bicarbonate active agent provided in Table 3 were prepared according to the procedure described in Example 1, except zinc oxide was replaced with sodium bicarbonate. Formulations 2A-2G include the ingredient amounts in base formulation 1A, except formulations 2A-2G include varying concentrations of kappa carrageenan as shown in Table 3. Formulations 2H-2N include the ingredient amounts in base formulation 1A, except formulations 2H-2N include varying concentrations of texturizer (i.e., StarDesign™ Power). Formulations 2O-2Q evaluate the effect of varying the concentration of sodium stearate in the presence of NaHCO₃ as compared to base formulation 1A. Formulations 2R-2T evaluate the ratio of sodium stearate to NaHCO₃ as provided in Table 3. Some water leaching was observed in formulations 2A-2T after 24 hours, indicating some instability in the formulations compared to formulation 1A.

TABLE 3
Natural-based deodorants with NaHCO₃ active

| | 2A | 2B | 2C | 2D | 2E | 2F | 2G | 2H | 2I | 2J | 2K |
|---|---|---|---|---|---|---|---|---|---|---|---|
| | [16A] | [16B] | [16C] | [16D] | [16E] | [16F] | [16G] | [16H] | [16I] | [16J] | [16K] |
| Ingredients (wt %) | | | | | | | | | | | |
| Water | 48.8 | 47.6 | 47.8 | 48.0 | 48.2 | 48.4 | 48.6 | 55.3 | 48.3 | 49.3 | 50.3 |
| Satiagel ™ VPC 614 | 0 | 1.2 | 1 | 0.8 | 0.6 | 0.4 | 0.2 | 1.5 | 1.5 | 1.5 | 1.5 |
| Tetrasodium EDTA | 0.1 | 0.1 | 0.1 | 0.1 | 0.1 | 0.1 | 0.1 | 0.1 | 0.1 | 0.1 | 0.1 |
| Sodium Stearate | 5.0 | 5.0 | 5.0 | 5.0 | 5.0 | 5.0 | 5.0 | 5.0 | 5.0 | 5.0 | 5.0 |
| Glycerin | 10.0 | 10.0 | 10.0 | 10.0 | 10.0 | 10.0 | 10.0 | 10.0 | 10.0 | 10.0 | 10.0 |
| NaHCO₃ | 2.0 | 2.0 | 2.0 | 2.0 | 2.0 | 2.0 | 2.0 | 2.0 | 2.0 | 2.0 | 2.0 |
| StarDesign ™ Power | 8.0 | 8.0 | 8.0 | 8.0 | 8.0 | 8.0 | 8.0 | 0.0 | 7.0 | 6.0 | 5.0 |
| High Oleic | 15.0 | 15.0 | 15.0 | 15.0 | 15.0 | 15.0 | 15.0 | 15.0 | 15.0 | 15.0 | 15.0 |
| Sunflower oil | | | | | | | | | | | |
| Hydrogenated | 3.0 | 3.0 | 3.0 | 3.0 | 3.0 | 3.0 | 3.0 | 3.0 | 3.0 | 3.0 | 3.0 |
| Coconut oil | | | | | | | | | | | |
| White Cocoa | 8.0 | 8.0 | 8.0 | 8.0 | 8.0 | 8.0 | 8.0 | 8.0 | 8.0 | 8.0 | 8.0 |
| Butter | | | | | | | | | | | |
| Vitamin E | 0.1 | 0.1 | 0.1 | 0.1 | 0.1 | 0.1 | 0.1 | 0.1 | 0.1 | 0.1 | 0.1 |

| | 2L | 2M | 2N | 2O | 2P | 2Q | 2R | 2S | 2T |
|---|---|---|---|---|---|---|---|---|---|
| | [16L] | [16M] | [16N] | [16O] | [16P] | [16Q] | [16R] | [16S] | [16T] |
| Ingredients (wt %) | | | | | | | | | |
| Water | 51.3 | 52.3 | 53.3 | 49.3 | 48.3 | 49.3 | 48.3 | 48.3 | 48.3 |
| Satiagel ™ VPC 614 | 1.5 | 1.5 | 1.5 | 1.5 | 1.5 | 1.5 | 1.5 | 1.5 | 1.5 |
| Tetrasodium EDTA | 0.1 | 0.1 | 0.1 | 0.1 | 0.1 | 0.1 | 0.1 | 0.1 | 0.1 |
| Sodium Stearate | 5.0 | 5.0 | 5.0 | 5.0 | 4.0 | 3.0 | 5.0 | 4.0 | 3.0 |
| Glycerin | 10.0 | 10.0 | 10.0 | 10.0 | 10.0 | 10.0 | 10.0 | 10.0 | 10.0 |
| NaHCO₃ | 2.0 | 2.0 | 2.0 | 2.0 | 2.0 | 2.0 | 1.0 | 2.0 | 3.0 |
| StarDesign ™ Power | 4.0 | 3.0 | 2.0 | 8.0 | 8.0 | 8.0 | 8.0 | 8.0 | 8.0 |
| High Oleic | 15.0 | 15.0 | 15.0 | 15.0 | 15.0 | 15.0 | 15.0 | 15.0 | 15.0 |
| Sunflower oil | | | | | | | | | |
| Hydrogenated | 3.0 | 3.0 | 3.0 | 3.0 | 3.0 | 3.0 | 3.0 | 3.0 | 3.0 |
| Coconut oil | | | | | | | | | |
| White Cocoa | 8.0 | 8.0 | 8.0 | 8.0 | 8.0 | 8.0 | 8.0 | 8.0 | 8.0 |
| Butter | | | | | | | | | |
| Vitamin E | 0.1 | 0.1 | 0.1 | 0.1 | 0.1 | 0.1 | 0.1 | 0.1 | 0.1 |

### Example 3: Comparative Natural-Based Deodorant Base Formulations

Comparative natural-based deodorants provided in Table 4 were prepared following the procedure described in Example 1.

TABLE 4

| | | Formulation (% conc) | | | | | |
|---|---|---|---|---|---|---|---|
| INCI | Trade Name | 3[3] | 4[5] | 5[9] | 6[11] | 7[13] | 8[14] |
| Water (aqua) | — | — | 66.7 | 52.2 | 51.6 | 27.3 | 46.8 |
| Carrageenan | Satiagel VPC 614 | — | — | 0.5 | — | — | — |
| (Kappa | | | | | | | |
| Carrageenan) | | | | | | | |
| Carrageenan (Iota | Satiagel VPC 508 | — | 3.0 | 2.0 | — | — | 3.0 |
| Carrageenan) | | | | | | | |
| Scleroglucan | Actigum ™ CS11 | — | — | — | 0.2 | — | — |
| Caprylic/Capric | Myritol 312 | 5.0 | — | — | — | — | — |
| Triglyceride | | | | | | | |
| Natural Fragrance | — | 1.0 | 0.25 | 0.25 | — | 1.5 | — |
| Lauryl Glucoside | Plantaren 1200 | — | — | — | — | 6.0 | — |
| Glycerin | Glycerin | — | 20.0 | 30.0 | 10.0 | 40.0 | 10.0 |
| Tetrasodium 60 | EDTA BX Pulver | — | — | — | 0.1 | 0.1 | 0.1 |
| EDTA | | | | | | | |
| Sodium Stearate | Sodium Stearate | — | — | — | 8.0 | 10.0 | 5.0 |
| | OP-200V | | | | | | |
| Sodium Starch | StarDesign ™ | — | — | — | 5.0 | — | 5.0 |
| Octenylsuccinate | Power | | | | | | |
| (n-OSA) and | | | | | | | |
| hydroxypropyl | | | | | | | |
| starch phosphate | | | | | | | |
| Hydroxypropyl | StarDesign ™ Care | — | 10.0 | 15.0 | — | — | — |
| starch phosphate | | | | | | | |
| Hydrogenated | RBD HYD 92 | — | | — | — | — | 5.0 |
| Coconut Oil | COC Oil KP Blk | | | | | | |
| Theobroma Cacao | Refined White | — | — | — | 5.0 | — | 5.0 |
| (Cocoa) Seed | Cocoa Butter | | | | | | |
| Butter | | | | | | | |
| Helianthus | High Oleic | 10.0 | — | — | 20.0 | — | 20.0 |
| Annuus | Sunflower Oil | | | | | | |
| Seed Oil | AP80 | | | | | | |
| Coconut Oil | Cocos nucifera | 48.9 | — | — | — | — | — |
| | (Coconut) oil | | | | | | |
| Candelilla Wax | Euphoribia | 5.0 | — | — | — | — | — |
| | Cerifera | | | | | | |
| | (candelilla) wax | | | | | | |
| D-Alpha | Vitamin E | 0.08 | 0.1 | 0.1 | 0.1 | 0.1 | 0.1 |
| Tocopheryl | | | | | | | |
| Acetate | | | | | | | |
| Denatured Alcohol | Alcohol | — | — | — | — | 15.0 | — |

The comparative natural-based deodorant formulations provided in Table 4 did not exhibit the balance of excellent processability, structural hardness, and good pay-off when compared to formulations in Examples 1 and 2 (see Tables 2 and 3). In particular, formulations 3 and 4 lacked stiffness necessary to mold the formulation into stick shapes, and formulation 4 also had a gelatinous texture. Formulation 5 exhibited issues with processing even under heated processing conditions, where the formulation was sticky and did not have good pay-off. Formulation 6 containing Scleroglucan as a gelling agent did not give the best texture and structure of the natural-based deodorant stick, as the formulation was too thick and chunky resulting in a sticky sensorial feel. Formulation 7 resulted in a natural-based deodorant stick that was off-balance, where the stick was too solid and exhibited poor pay-off with a sticky, tacky, and oily sensorial feel. Formulation 8, which included iota carrageenan as the gelling agent with StarDesign™ Power as texturizer, exhibited a poor pay-off that resulted in large chunks of product being left on the skin upon application.

### Example 4: Cone Penetration Evaluation

To further understand and evaluate the hardness/softness of the exemplary natural-based deodorant formulations, a cone penetration testing was done on the following exemplary formulations and commercial market deodorant products according. ASTM Standard Test Method for Cone Penetration of Petrolatum was used as a reference and adapted for testing the commercial samples and the lab prototypes. [ASTM: Designation: D 937-97 (Reapproved 2002) Designation: 179/79 (95) An American National Standard British Standard 4698 Standard Test Method for Cone Penetration of Petrolatum]

The formulation/technology of each commercial sample is different based on the ingredient INCI list printed on the products. The cone penetration analysis compares the natural-based deodorant formulations in keeping with present technology (e.g., Examples 1-3) to different structures and textures of the commercial samples.
Exemplary Formulations: 1B, 1E, 1F, 1G, and 1I;
Comparative Formulations: 3, 4, 5, 6, 7, and 8
Commercial Samples: Schmidt's, Native, Axe, Degree, Tom's of Maine, Old Spice, and Speed Stick

Summary of Test Method: The commercial samples were melted to a completely liquid state and then cooled to the lab ambient temperature of 23° C. The penetration is measured with the cone and the sample at this temperature using a penetrometer by means of which a standard cone is applied to the sample for 5 s under a load of 150 g. Deodorants come in varying texture and consistency. Some are a firm gel, whilst others are a soft solid. Cone penetration is a means of measuring the firmness or consistency of deodorant. Such measurements are useful for selecting, specifying and comparing various of deodorant samples for consistency or firmness. Cone penetration values may or may not correlate with end-use functional properties.

Apparatus: 1. Penetrometer and Cone, as specified in Test Methods D 217.2. Containers, cylindrical, having a flat bottom 96 mm in diameter and 65 mm or more in depth, constructed of at least 1 mm metal and provided with a well-fitting water-tight cover.

Procedure: 1. The surface of the sample was not cut to level the sample or worked in any other way, as this would affect the result. 2. Samples were placed in a container and on the penetrometer table located such that the tip of the penetrometer cone is 25 to 40 mm in from the rim of the container. The cone was observed in its “zero” position and adjustment of either the indicator assembly or the table, dependent on the type of instrument, was done until the tip of the cone just touched the surface of the sample. The plunger was quickly released and held for 5 s. Total penetration was read from the scale. Measurements were performed in triplicate for each sample as further described in steps 3 and 4.

3. With samples having penetrations less than 200, three tests (and sometimes more) may be made in one container by proper spacing. To prevent one test from being affected by the disturbed area of a previous test, the tip of the cone was not placed close to the edge of a previous test area than the penetration distance of the sample.

4. With samples having penetrations over 200, only one test was made in a container and the cone tip was placed as exactly as possible at the center of the container.

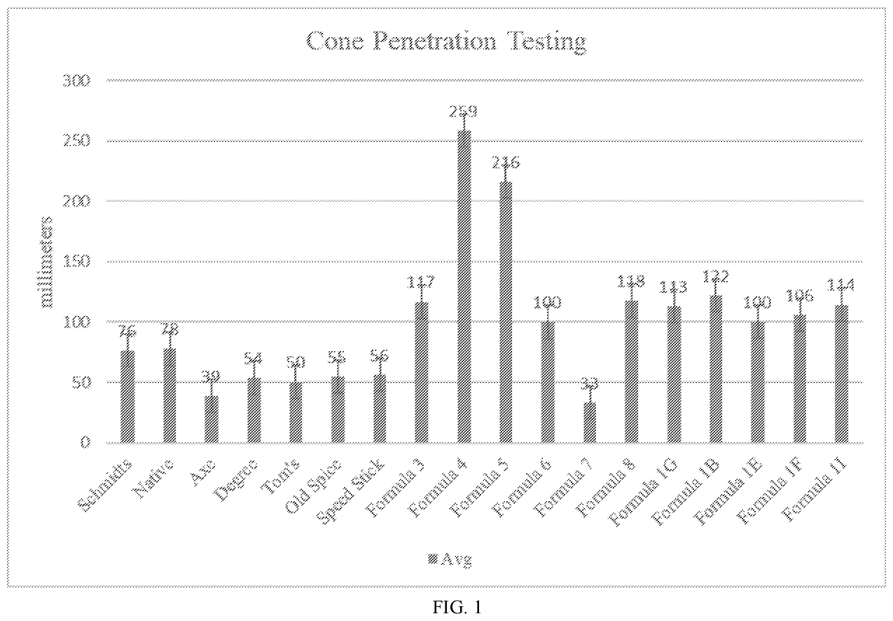

Report: 1. As shown in FIG. 1, the average of all results was reported to the nearest 1.0 mm as the penetration, ASTM D 937 (1998). Test Variations: Samples are deodorants and not petrolatum. Commercial samples were melted on a hot plate (not an oven) till a completely liquid (not to a certain temperature of 82° C.). Exemplary formulations of the present technology were made according to procedure described in Example 1 and directly poured in sample jars. Samples were held at an ambient lab temperature of 23° C. for greater than 18 hours, (not 25° C.). Sample were not subjected to a water bath of 25° C. for 2 hours. Minimum of 3 readings was measured for each sample. In the event of a significant If one of the three measurements varied greatly for the others, a fourth measurement was preformed, and the outlier was disregarded. Averages of the measurements were reported to 1 mm and (not to 0.1 mm).

The commercial samples showed results ranging from 39-78 mm (FIG. 1), indicating firmer structures. In contrast, the formulations 1B, 1E, 1F, 1G, 1I, and 3-8 were relatively less firm with results ranging from 100-259 mm. Formulation 7 was an exception with 33 mm, indicating very firm structure.

Formulations 4 and 5 exhibited cone penetration values of 259 mm and 216 mm, respectively, which indicated a very soft texture that lacked stiffness and lacked sensory due to some stickiness and poor pay-off. Formulations 3 and 6 exhibited suitable cone penetration values of 117 mm and 100 mm, respectively, but these formulations were not desirable with respect to processing and sensorial benefits. Formulations 1B, 1D, 1E, and 1I exhibited cone penetration values in the range of 100-125 mm (FIG. 1), corresponding to the sensory feel that is desirable in terms of a balanced structure with good pay-off and non-stick, non-tacky skin feel, with respect to formulations containing Satiagel™ VPC 614, sodium stearate, and StarDesign™ Power (SDP) texturizer.

Based on the results from the cone penetration testing, it can be concluded that the concentration of Satiagel™ VPC 614 exhibited a dose dependent impact on structure. As seen in formulations 1B, 1G, and 1E with 1.0%, 1.5% and 2.0%, respectively, of Satiagel™ VPC 614 showed cone penetration values of 122 (firmer), 113 (less firm) and 100 mm (less firm), respectively. Whereas with formulations 1F (6% SDP), 1G (8% SDP), 1I (10% SDP), did not show much difference in cone penetration readings at 106, 113 and 114 mm. This indicated that SDP in the range of 6-10% gives similar structural profile and does not show dose dependent impact in the formulations.

The present examples demonstrate the present technology successfully provides natural-based deodorant sticks having excellent functional and performance properties-such as, sensorial benefits, structural integrity, and good pay-off. Cone penetration tests confirmed the perceived sensory feel and structural hardness of the exemplary formulations. Accordingly, the exemplary formulations provide that provide a cleaner, minimalist natural-based deodorant sticks giving good functional properties of easy spreading, a non-sticky/tacky skin feel and smooth application of the product.

Each of the non-limiting aspects above can stand on its own, or can be combined in various permutations or combinations with one or more of the other aspects or other subject matter described in this document. While certain aspects have been illustrated and described, a person with ordinary skill in the art, after reading the foregoing specification can effect changes, substitutions of equivalents and other types of alterations to the present technology as set forth herein. Each aspect described above can also have included or incorporated therewith such variations or aspects as disclosed in regard to any or all of the other aspects and aspects.

The present technology is also not to be limited in terms of the particular aspects described herein, which are intended as single illustrations. Many modifications and variations of this present technology can be made without departing from its spirit and scope, as will be apparent to those skilled in the art from the foregoing descriptions. Such modifications and variations are intended to fall within the scope of the appended claims. It is to be understood that this present technology is not limited to particular methods, reagents, compounds, or compositions, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The aspects, illustratively described herein may suitably be practiced in the absence of any element or elements, limitation or limitations, not specifically disclosed herein. Thus, for example, the terms “comprising.” “including.” “containing,” etc. shall be read expansively and without limitations. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

1. A natural-based deodorant composition comprising:
about 5.0 weight percent (wt %) to about 15.0 wt % of a texturizer based on total weight of the deodorant composition;
about 0.5 wt % to about 2.5 wt % of a gelling agent comprising kappa carrageenan; and
about 5.0 wt % to about 35.0 wt % of an emollient composition; and
water;
wherein the texturizer comprises:
a n-octenyl succinate anhydride (nOSA) modified starch; and
a hydroxypropylated starch, wherein a weight ratio of the nOSA modified starch to hydroxypropylated starch is about 3:1 to about 20:1.

2. The deodorant of claim 1, wherein the deodorant composition comprises about 5.5 to about 12.5 wt % of the texturizer.

3-4. (canceled)

5. The deodorant of claim 1, wherein the weight ratio of nOSA modified starch to hydroxypropylated starch is about 3:1 to about 10:1.

6. The deodorant of claim 5, wherein the nOSA modified starch has a degree of substitution of about 0.1% to about 3.0%.

7. The deodorant of claim 5, wherein the texturizer comprises about 75 wt % to about 95 wt % of the nOSA modified starch based on total weight of the texturizer.

8. The deodorant of claim 1 comprising about 1.0 wt % to about 2.3 wt % of the gelling agent.

9. (canceled)

10. The deodorant of claim 1, wherein the emollient composition comprises an edible solid fat, an edible liquid fat, or combinations thereof.

11. The deodorant of claim 10, wherein the emollient composition comprises a weight ratio of the edible solid fat to the edible liquid fat of about 1:1 to about 1:3.

12-14. (canceled)

15. The deodorant of claim 1 further comprising one or more additives comprising thickeners, humectants, antioxidants, antimicrobial agents, chelating agents, colorants, fragrances, additional emollients, conditioning agents, butters, waxes, starches, gums, clays, essential oils, plant extracts, odor absorbers, or combinations thereof.

16. The deodorant of claim 15, wherein the deodorant further comprises one or more of:
thickeners selected from the group consisting of sodium stearate, lithium stearate, aluminum stearate, aluminum distearate, aluminum tristearate, calcium stearate, magnesium stearate, potassium stearate, zinc stearate, or combinations thereof;
humectants selected from the group consisting of glycerin, sorbitol, propylene glycol, triethylene glycol, tripropylene glycol, polypropylene glycol, butylene glycol, urea, aloe vera juice, and combinations thereof;
antioxidants selected from the group consisting of vitamin E, vitamin A, vitamin C, butylated hydroxytoluene (BHT), butylated (BHA), natural plant extracts, and combinations thereof; or
chelating agents selected from the group consisting of EDTA, citric acid, sodium phytate, gluconodelta lactone, and combinations thereof.

17-21. (canceled)

22. The deodorant of claim 1, wherein the deodorant has a cone penetration of about 90 mm to about 140 mm.

23. (canceled)

24. The deodorant of claim 1 further comprising an active agent comprising zinc oxide, sodium bicarbonate, triclosan, salicylic acid, triclocarbon, quaternary ammonium compounds, ammonium salts, zirconium salts, glycols, or combinations thereof.

25. (canceled)

26. The deodorant of claim 1, wherein the deodorant exhibits one or more improved functional properties comprising excellent processability, good pay-off, easy spreadability, improved skin feel, or combinations thereof.

27. The deodorant of claim 1, wherein the hydroxypropyl starch is a hydroxypropyl starch phosphate.

28. A method for stabilizing a cosmetic deodorant composition comprising:
preparing a first mixture comprising a gelling agent comprising kappa carrageenan and water;
contacting the first mixture with a texturizer;
preparing a second mixture comprising an emollient composition, wherein the emollient composition comprises an edible solid fat and an edible liquid fat;
heating the first mixture and the second mixture to a temperature of about 65° C. to about 85° C.; and
combining the first mixture and the second mixture to obtain a stabilized cosmetic deodorant;
wherein the texturizer comprises:
a n-octenyl succinate anhydride (nOSA) modified starch; and
a hydroxypropylated starch, wherein a weight ratio of the nOSA modified starch to hydroxypropylated starch is about 3:1 to about 20:1.

29. The method of claim 28, wherein the first mixture further comprises one or more additives comprising thickeners, humectants, antioxidants, antimicrobial agents, chelating agents, colorants, fragrances, additional emollients, conditioning agents, butters, waxes, starches, gums, clays, essential oils, plant extracts, odor absorbers, or combinations thereof.

30. The method of claim 28, wherein the first mixture further comprises an active agent comprising zinc oxide, sodium bicarbonate, triclosan, salicylic acid, triclocarbon, quaternary ammonium compounds, ammonium salts, zirconium salts, glycols, or combinations thereof.

31. (canceled)

32. The method of claim 28, wherein the cosmetic deodorant has a cone penetration of about 90 mm to about 140 mm.

33. The method of claim 28, wherein the cosmetic deodorant exhibits one or more improved functional properties comprising excellent processability, good pay-off, easy spreadability, improved skin feel, or combinations thereof.
